(19) United States
(12) Patent Application Publication
(10) Pub. No.: US 2014/0202467 A1
(43) Pub. Date: Jul. 24, 2014

(54) Condom cover shield

(71) Applicant: Daniel James Downey, El Dorado Hills, CA (US)

(72) Inventor: Daniel James Downey, El Dorado Hills, CA (US)

(21) Appl. No.: 13/694,913

(22) Filed: Jan. 19, 2013

(51) Int. Cl. A61F 6/04

(52) U.S. Cl. CPC A61F 6/04
USPC 128/844

(57) ABSTRACT

The Condom Cover Shield (CCS) is a flexible tube that is fitted over a condom during the condom manufacturing process. This protective closed end tube is rolled up with the new condom therefore allowing the condom/shield combination to be installed onto the penis in the same manner a condom alone is installed. Once the condom/shield is installed the shield is slipped off leaving an installed condom with an untouched exterior surface. A lubricant will be placed between the condom and cover shield to allow for easy removal of the shield after installation.

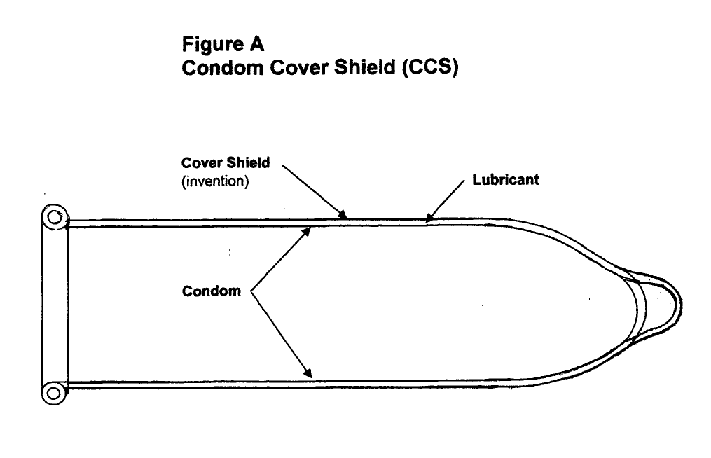

### CROSS-REFERENCE TO RELATED APPLICATIONS

Provisional Application No. 61/633,065—Filed Feb. 6, 2012 Confirmation No. 6661

### STATEMENT REGARDING FEDERALLY SPONSORED RESEARCH OR DEVELOPMENT

Not Applicable

### BACKGROUND OF THE INVENTION

The field of endeavor is to provide protection from the spread of sexually transmitted diseases during the process of installing a condom on to a penis. This device should reduce the chance of contaminating the outside of an installed condom with pre-ejaculation fluid. Numerous articles exist on the fact that pre-ejaculation fluid can contain HIV virus and male sperm. This device should also reduce the unwanted spread of sperm onto the outside of an installed condom.

### BRIEF SUMMARY OF THE INVENTION

The Condom Cover Shield is a flexible tube that is fitted over a condom during the condom manufacturing process. This protective tube is rolled up with the new condom therefore allowing the condom/shield combination to be installed onto the penis in the same manner a condom alone is installed. Once the condom/shield is installed the shield is slipped off leaving an installed condom with an untouched exterior surface.

### BRIEF DESCRIPTION OF THE VIEW OF THE DRAWING

Only one drawing is submitted with this application. The attached drawing shows a standard type condom, with and without an extended end, covered by a second tube, the condom cover shield. Also depicted is lubricant between the condom and the cover shield so that the shield can be removed easily without touching the condom.

### DETAILED DESCRIPTION OF THE INVENTION

The Problem

During the installation of condoms on to the penis the installer's fingers roll over the top of the condom ring therefore touching the skin of the penis. During sexual interaction with a partner, it is likely that the penis skin has been contaminated with bodily fluid from the penis during foreplay petting by the male's partner. Pre-come/pre-ejaculation fluid excreted by the penis during foreplay may contain sperm and/or STDs (sexually transmitted disease). This fluid is usually deposited over the penis by the partner during foreplay activities. This rolling/touching activity may happen many times during the condom installation, therefore transmitting fluid from the penis skin to the exterior of the condom as the male returns his fingers in front of the role to continue the condom installation process. This continued process may contaminate the exterior of the condom numerous times during the installation, therefore providing opportunity for male bodily fluid to be transported to the internal cavities of his partner during sexual activity.

The Solution—Condom Cover Shield

The Condom Cover Shield is a flexible tube that is fitted over a condom during the condom manufacturing process. This protective tube is rolled up with the new condom therefore allowing the condom/shield combination to be installed onto the penis in the same manner a condom alone is installed. A lubricant will most likely be needed to be applied to the condom prior to the installation of the shield. This would allow for easy removal of the shield after the condom/shield combination is installed. Once the condom/shield is installed the shield is slipped off leaving an installed condom with an untouched exterior surface.

1. The subject matter of this claim is a flexible closed end tube that covers a condom until the condom is installed onto a penis or other tubular item (dildow, vibrator, etc.) Once the condom/cover shield combination is installed on a penis or other object, the cover shield is slipped off the condom leaving an untouched condom exterior.

2. A Condom Cover Shield (CCS) is to protect the exterior of a condom from becoming contaminated with any liquids that may contain sexually transmitted diseases and/or sperm during the condom installation procedure. Contamination of the outside of a condom may occur during installation when the installers fingers roll over the condom roll, touching the penis skin and then returning to the outside of the condom to continue condom installation. The Condom Cover Shield will protect the exterior of the condom when roll over occurs and installation continues. Once installed, this shield is removed without touching the exterior of the condom, leaving the condom untouched and uncontaminated just prior to intercourse. The condom should not be touched by anyone until intercourse is completed.
